(19) United States
(12) Patent Application Publication
(10) Pub. No.: US 2021/0322314 A1
(43) Pub. Date: Oct. 21, 2021

(54) Use Of Liposomes To Deliver A Protein And A Gene Encoding The Protein To A Live Cell

(71) Applicants: Avinash SETH, Pomona, CA (US); Robert D. FISH, Tustin, CA (US); So Young Life Sciences Corporation, San Dimas, CA (US)

(72) Inventors: Avinash SETH, Pomona, CA (US); Robert D. Fish, Tustin, CA (US)

(21) Appl. No.: 17/259,910

(22) PCT Filed: Jul. 1, 2019
(86) PCT No.: PCT/US2019/040156

§ 371 Date: Jan. 12, 2021

Related U.S. Application Data
(60) Provisional application No. 62/702,482, filed on Jul. 24, 2018.

(51) Int. Cl. A61K 9/127; A61P 35/00; C12N 15/86; A61K 38/17; A61K 39/395

(52) U.S. Cl. CPC A61K 9/127; A61P 35/00; C12N 2710/16143; A61K 38/1709; A61K 39/3955; C12N 15/86

(57) ABSTRACT

The present invention provides compositions and methods for treating a cancer using liposomes. Collectively, the liposomes contain a protein and a vector encoded for a gene corresponding to the protein. The amount of the protein and the vector a plurality of liposome is sufficiently effective to treat cancer cells. At least some of the liposomes can contain an antibody that recognizes, and thereby targets, a protein expressed on the cancer cell.

### PRIORITY CLAIM

This application claims priority to U.S. provisional application Ser. No. 62/702482, filed Jul. 24, 2018. The '482 application, and all other referenced extrinsic materials are incorporated herein by reference in their entirety. Where a definition or use of a term in a reference that is incorporated by reference is inconsistent or contrary to the definition of that term provided herein, the definition of that term provided herein is deemed to be controlling.

### FIELD OF THE INVENTION

The field of the invention is compositions and methods for delivering a protein and a gene of the protein into a live cell by use of liposome.

### BACKGROUND

The following background description includes information that may be useful in understanding the present invention. It is not an admission that any of the information provided herein is prior art or relevant to the presently claimed invention, or that any publication specifically or implicitly referenced is prior art.

There are significant pharmaceutical and biopharmaceutical issues involved with delivery of peptides, proteins, and other biotech molecules to a patient. One of the common approaches to overcoming these issues is to use liposomes as carriers. Unfortunately, liposome delivery of a protein is often inefficient, and fails to effectively target the cells of interest. In addition, even if proteins are delivered to, and then transported into the cells, proteases within the cytosol can rapidly degrade the proteins.

Proteins can also be provided to cell by transfecting the cells with a genetic sequence, and causing the cells to express a corresponding protein. Genes can be readily delivered by calcium phosphate transfection, however, the efficiency is usually very low, only about 10%. Genes can be delivered with higher efficiency using viruses, but there are serious issues in using this approach with a live human. And, of course, even where transfection is accomplished, the cell takes time to express significant amounts of the corresponding protein.

Therefore, there are still difficulties in providing specified proteins to cells for therapeutic or other purposes, especially where the cells are part of a living person or animal.

### SUMMARY OF THE INVENTION

The inventive subject matter provides compositions and methods for delivering proteins to cells of living persons or animals, using a combination of exogenous and endogenous delivery systems. Using that combination, the exogenously produced protein is delivered rapidly to the cells, during which time the cells can gear up to endogenously express other copies of the protein.

In preferred embodiments, the exogenous delivery is accomplished using liposomes, which include a protein that had been expressed by bacteria or yeast. Contemplated vectors to transfect the bacteria or yeast include pCMVA6-AC, a vector that contains a strong promoter.

Also in preferred embodiments, the endogenous delivery of protein is accomplished using liposomal delivery, but in this case by including within the liposome, a vector that includes the gene that encodes the protein.

The same liposomes that include the protein can be, but need not be, the same liposomes that include the gene. Regardless, to enhance efficiency of delivery of the liposomes the liposomes can include on their surfaces, a target-specific antibody, for example, ESK1 (Recombinant human anti-WT1 antibody available from Creative Biolabs™) to target cancer cells.

### BRIEF DESCRIPTION OF THE DRAWINGS

FIG. 1 is a flow chart illustrating one embodiment of the process delivering a protein and its sequence into a live cell.

FIG. 2 illustrates a diagram of the pCMV6-AC-GFP tagged cloning vector, (available from OraGene™) , which contains the Smad4 gene.

FIG. 3 illustrates an electrophoretic result of the protein expression (Smad4).

FIG. 4 illustrates a graph showing apoptotic effect of the protein (Smad4).

FIG. 5 illustrates a graph showing proliferative effect of the protein (Smad4).

### DETAILED DESCRIPTION

Liposomes have been widely used to introduce exogenous molecules into cells for pharmaceutical purposes. There still remain, however, unresolved issues associated with delivering therapeutically effective amounts of such molecules into target cells. The present invention herein addresses that problem by using liposomes to deliver both a protein and its genetic sequence to target cells.

Of particular interest is targeting cancer cells, including especially breast and pancreatic cancer cells.

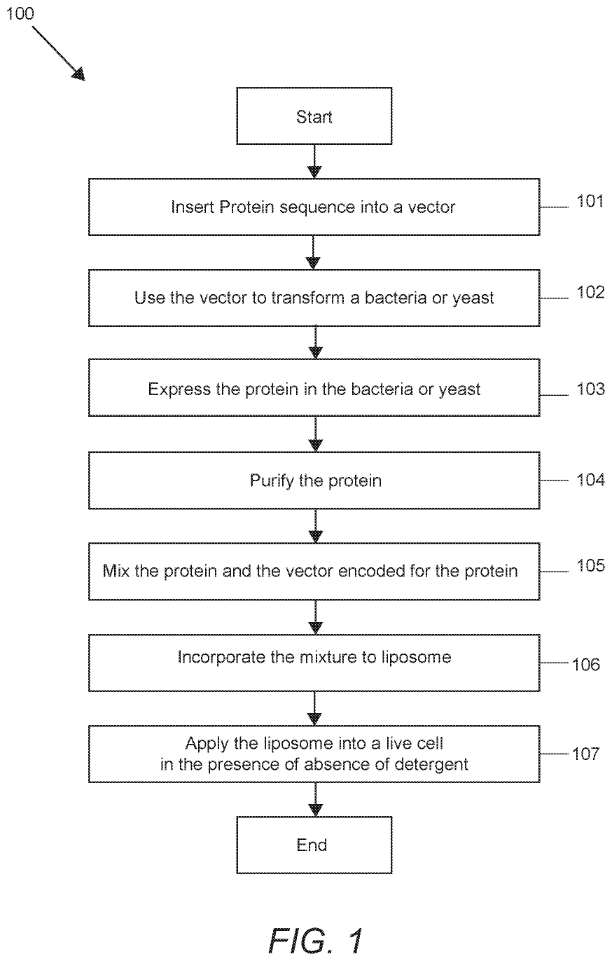

FIG. 1 is a flow chart illustrating preferred steps in delivering a protein and its sequence into a live cell. For protein preparation, a vector encoded with sequence of the protein 101 is used to transform a bacteria or yeast 102. The protein is expressed in bacteria or yeast 103, and is then purified 104 by use of conventional methods. The purified protein is mixed with the vector encoded with the sequence of the protein 105, the mixture 106 is includes within a liposome, and the liposome is applied to a live cell in the presence or absence of detergent 107. The detergent is added to the cell to induce efficiency for liposome endocytosis into the cell.

Any suitable vector can be used. Preferred vectors include bacterial, yeast or mammalian vectors. The bacterial and yeast vectors are useful vectors to express a protein in bacteria or yeast wherein the vector encodes the sequence of the protein. Whereas mammalian vectors are required for mammalian cells to express a protein in which the genetic sequence of the protein is encoded in the vector. In addition, the vector can contain a strong promoter (constitutively active). The vector used for protein expression in the bacteria or yeast can be the same or different from the one included in the liposome. Similarly, it is also contemplated that instead of mixing the protein and the vector with a given liposome, protein and vectors could be enclosed in different liposomes. In that latter case, the different liposomes could be co-administered, or administered in sequence, under a protocol in which the exogenously provided protein is present in the target cells concurrently with the protein being expressing endogenously as a result of the target cells being transfected with the vector.

In preferred embodiments the protein expressed in the bacteria will be the same protein coded by the vector included in the liposome. The combination of these two allows to have the protein in cells at the beginning of liposome administration due to direct protein delivery of the protein and the protein is continuously expressed later because the transcriptional factor of the protein is expressed. Alternatively, it is contemplated that the two proteins could be different. For example, the vector included in the liposome could contain a genetic sequence of a transcriptional factor, and the protein expressed in the bacteria could be the protein whose expression is induced by the transcription factor.

Administration of the liposomes can occur by known means, including intravenous, intramuscular, oral, topical, transdermal, transmucosal, and iontophoretic deliveries.

### Experiment

In order to observe whether this method works sufficiently well to produce physiologically significant results, experiments were undertaken with a protein called Smad4 . Smad4 is well known as a tumor suppressive protein, which induces cell apoptosis and inhibits cell proliferation. Accordingly, cell apoptosis and proliferation were observed in cancer cells to ascertain effectiveness of the method. Additional details are included in the attached manuscript.

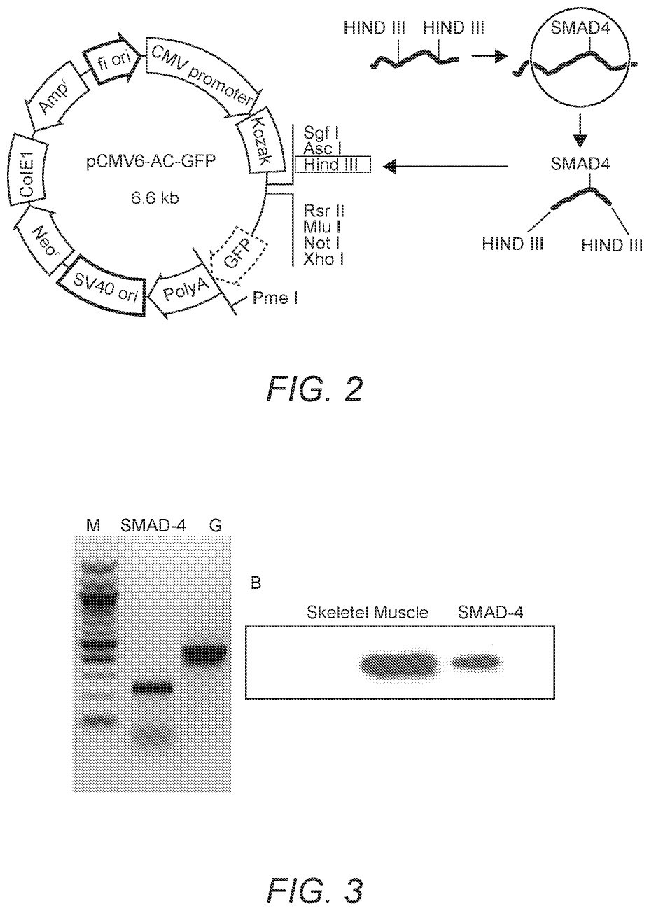

FIG. 2 shows a vector design containing the gene of Smad4 (AAM74472). The vector used herein contains a sequence of GFP (green fluorescent protein), such that the expression of Smad4 can be detected under fluorescence observation.

FIG. 3 in an image of a Western blot that depicts expression of Smad4 protein in breast cancer cells, using the protocol of FIG. 1. In this experiment, a mixture of Smad4 protein and Smad4 generic sequence were added to liposome contained an ESK1 antibody, and the liposomes were used to target breast cancer cells. The breast cancer cells were obtained from transgenic mice which carry cells from the NF639 breast cancer cell line. The cells were lysed and RT-PCR was performed to detect the mRNA expression (middle column) of the Smad4 sequence. The left column shows a corresponding molecular weight ladder, and the right column shows expression of the Smad4 protein. The blot shows that the Smad4 gene was delivered to the nucleus of the cells, and the cells are producing the Smad4 protein. By itself, FIG. 3 does not rule out the possibility of endogenous Smad4 production in the absence of transfection via the liposomes.

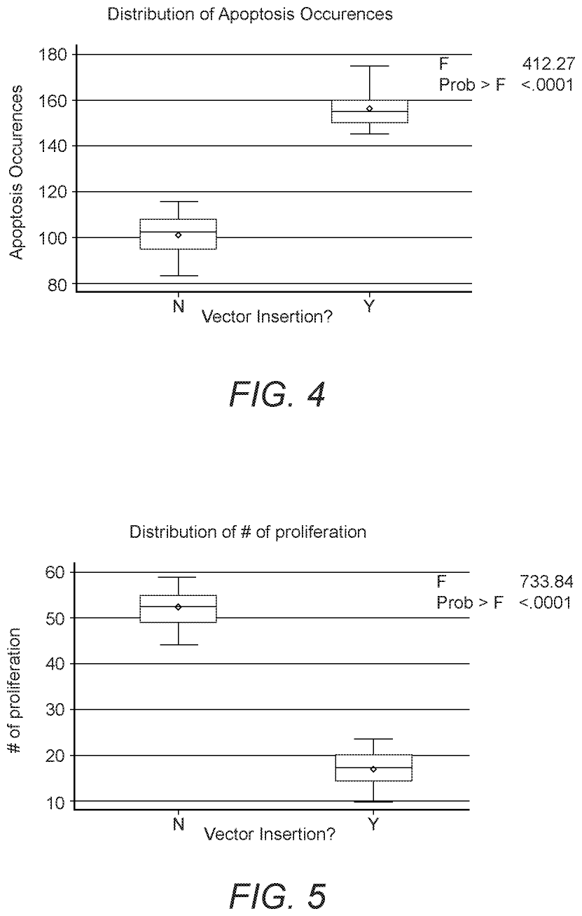

FIG. 4 is a graph comparing the apoptosis in breast cancer cells (a) using liposomes with a vector having a Smad4 genetic sequence with (b) that of similar cells using liposomes with a NULL vector. Since presence of Smad4 protein is positively correlated with apoptosis, this data demonstrates that the cells transfected using liposomes with a vector having the Smad4 sequence expressed significantly more Smad4 protein than those with the NULL vector. Accordingly, it was concluded that the liposomes with a vector having a Smad4 genetic sequence were effective in the transfected cells with that sequence.

FIG. 5 is a graph comparing the proliferation in breast cancer cells (a) using liposomes with a vector having a Smad4 genetic sequence with (b) that of similar cells using liposomes with a NULL vector. Since presence of Smad4 protein is negatively correlated with proliferation, this data demonstrates that the cells transfected using liposomes with a vector having the Smad4 sequence expressed significantly more Smad4 protein than those with the NULL vector. Accordingly, it was concluded that the liposomes with a vector having a Smad4 genetic sequence were effective in the transfecting cells with that sequence.

Additional data is forthcoming to demonstrate effectiveness of liposomes that include both exogenous Smad4 and a vector having a Smad4 genetic sequence.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

1.-20. (canceled)

21. A composition for treating a cancer, the composition comprising:
a first amount of a protein;
a second amount of a vector containing a gene corresponding to the protein; and
wherein the first and second amounts are collectively contained in a plurality of liposomes, and wherein the first and second amounts are sufficient effective to treat the cancer.

22. The composition of claim 1, wherein the plurality of liposomes collectively further comprise an antibody configured to a specific target cell of the cancer.

23. The composition of claim 2, wherein the antibody comprises ESK1.

24. The composition of claim 2, wherein the specific target cell is a cancer cell.

25. The composition of claim 2, wherein the specific target cell is a breast cancer cell.

26. The composition of claim 2, wherein the specific target cell is a pancreatic cancer cell.

27. The composition of claim 1, wherein the vector comprises pCMV6-AC.

28. The composition of claim 1, further comprising a detergent.

29. The method of claim 1, wherein the protein comprises Smad4.

30. The composition of claim 1, wherein at least some of the protein and at least some of the vector are contained in a single liposome of the plurality of liposomes.

31. The composition of claim 1, wherein at least some liposomes of the plurality of liposomes contain amounts of the protein but do not contain the vector.

32. A method of increasing a specified protein in a target cell, comprising:
expressing the specified protein using a producing cell;
extracting the specified protein from the producing cell to generate an extracted protein;
packaging in a first liposome, a first amount of the extracted protein;
packaging in a second liposome, a second amount of a vector encoded with a gene configured to express the specified protein; and
targeting the target cell with the first and second liposomes.

33. The method of claim 12 wherein each of the first and second liposomes contain the extracted protein and the vector.

34. The composition of claim 12, wherein the first liposome contains the extracted protein but do not include the vector.

35. The method of claim 12, wherein at least one of the first and the second liposomes includes an antibody configured to target the target cell.

36. The method of claim 15, wherein the antibody comprises ESK1.

37. The method of claim 15, wherein the vector comprises pCMV6-AC.

38. The method of claim 15, wherein the protein comprises Smad4.

39. The method of claim 15, wherein the antibody comprises ESK1, the vector comprises pCMV6-AC, and the specified protein comprises Smad4.

40. The method of claim 12, further comprising delivering a detergent along with the first and the second liposomes.
